(19) United States
(12) Patent Application Publication
(10) Pub. No.: US 2024/0164864 A1
(43) Pub. Date: May 23, 2024

(54) Bone Flap Stabilizer Tool for Cranial Surgery

(71) Applicant: Zaid Aljuboori, Wilkes Barre, PA (US)

(72) Inventor: Zaid Aljuboori, Wilkes Barre, PA (US)

(21) Appl. No.: 18/188,204

(22) Filed: Mar. 22, 2023

Related U.S. Application Data
(60) Provisional application No. 63/384,408, filed on Nov. 18, 2022.

(51) Int. Cl. A61B 90/14

(52) U.S. Cl. CPC A61B 90/14

(57) ABSTRACT

A tool for stabilizing a bone flap during a craniotomy or the like provides a screw and head for controlled insertion through a boar in a central location of the bone flap and a handle extending outwardly there from for installation of the screw and subsequent retention of the bone flap against slipping from the surgeon's grip and stabilization of the bone flap against rotation and tilting.

### CROSS REFERENCE TO RELATED APPLICATION

This application claims the benefit of U.S. provisional application 63/384,408 filed Nov. 18, 2022 and hereby incorporated by reference.

### STATEMENT REGARDING FEDERALLY SPONSORED RESEARCH OR DEVELOPMENT

### BACKGROUND OF THE INVENTION

The present invention relates to surgical procedures, such as craniotomies, where a portion of the skull is removed and, in particular, to a stabilizing instrument for assisting the surgeon during such procedures.

During cranial surgery an opening in the skull may be made by removing a disk from the skull, termed a bone flap. This removal may entail drilling a set of burr holes around the periphery of the bone flap and introducing a special saw called a craniotome to complete the release of the bone flaps from the remainder of the skull. After separation, the bone flap is lifted away from the skull to expose the protective covering of the brain, called the dura, which may be pulled back to reveal the brain. At the completion of surgery, the bone flap is replaced into the skull and reattached using fasteners such as titanium screws, chemical bonding agents, or plates which may also fill the burr holes. Finally, the muscles and skin over the bone flap are sutured back together.

### SUMMARY OF THE INVENTION

The present invention addresses a problem presented by the sharp edges of the bone flap which, when tipped, may cut into the dura, brain, or surgeon's gloves during the removal process, the latter breaching the sterility of the surgical field. Avoiding the sharp edges of the bone flap by grasping the smooth outer surface of central region of the bone flap increases a risk of dropping the bone flap during this procedure.

In order to address these and other problems, the present invention provides a stabilizing handle that may be releasably attached to a central location of the bone flap to allow improve stabilization of the bone flap edges against tipping and rotation without contacting the bone flap edges. The stabilizing handle also provides an improved application of force at a central portion of the bone flap reducing tipping and the risk of dropping the bone flap. In one embodiment, a screw portion of the handle may be retained in the bone flap after surgery to substantially reduce disassembly time and to eliminate the need to fill or cover this additional skull opening.

Specifically then, in one embodiment, the invention may provide a surgical tool for stabilizing a bone flap during removal or attachment to a skull, the tool including: a screw having a threaded portion terminating at a head portion, the threaded portion sized to be received through a bore in a central location in the bone flap to engage the bone flap to a depth controlled by a head portion abutting an outer surface of the bone flap; and a stabilizing handle attaching to the screw to apply a torque to the screw to thread the threaded portion through the bore and to be retained on the screw to extend outwardly therefrom to a distal grip portion allowing stabilization of the bone flap during surgery.

It is thus a feature of at least one embodiment of the invention to provide for positive control of the bone flap during surgery at a location removed from regions in which the bone flaps must be cut or reattached to the skull and the resulting sharp edges. It is another feature of one embodiment to provide improved leverage on the bone flap to ensure its stabilization against tipping.

The depth of the threaded portion beneath the screw head may be limited to be less than 10 mm.

It is thus a feature of at least one embodiment of the invention to provide a screw that is self-limiting in depth of installment to protect the brain and dura.

The threaded portion may have an outer diameter from 3 to 6 mm.

It is thus a feature of at least one embodiment of the invention to provide a sufficient gripping interface between the screw and the material of the skull to ensure the necessary stabilization.

The screw may be a radiolucent material.

It is thus a feature of at least one embodiment of the invention to allow the screw to be retained in the skull after the procedure eliminating an additional disassembly step and the need to cover or fill an additional hole in the bone flap.

The screw may be constructed of a material from the group consisting of a polymer and a ceramic.

It is thus a feature of at least one embodiment of the invention to provide a biocompatible screw for long-term retention in the body with reduced interference in medical imaging procedures.

The head portion of the screw may include a second threaded portion attachable to corresponding threads of a proximal end of the stabilizing handle with rotation of the distal grip portion.

It is thus a feature of at least one embodiment of the invention to provide a simple yet robust attachment between the screw and handle allowing the handle to be removed at the conclusion of the procedure.

The surgical tool may further include a drive engaging with the screw head and rotatable independently of the stabilizing handle for applying a torque to the screw.

It is thus a feature of at least one embodiment of the invention to provide independent features for the transmission of torque and the retention of the screw and handle to optimize these different functions.

The drive and stabilizing handle may be coaxial.

It is thus a feature of at least one embodiment of the invention to provide a single tool that may both attach to the screw and independently provide torque for engaging and disengaging the screw.

These particular objects and advantages may apply to only some embodiments falling within the claims and thus do not define the scope of the invention.

### BRIEF DESCRIPTION OF THE DRAWINGS

FIG. 1 is a simplified perspective view of a patient's head during a craniotomy showing removal of a bone flap as stabilized by a surgical tool per the present invention;

FIG. 2 is a fragmentary elevational cross-section through the bone flap of FIG. 1 holding a previously inserted screw element whose depth is controlled by a screw head abutting an outer surface of the bone flap;

FIG. 3 is a perspective view of a handle attachable to the screw of FIG. 2 and showing, as an inset, the coaxial drive and threaded attachment elements;

FIG. 4 is an alternative embodiment of an attachment element of FIG. 3 using external threads and an internal torque drive;

FIG. 5 is an alternative embodiment to those of FIGS. 3 and 4 combining the torque and attachment features; and

FIG. 6 is a cross-sectional view similar to that of FIG. 2 showing a tool employing a permanent attachment between the handle and screw.

### DETAILED DESCRIPTION OF THE PREFERRED EMBODIMENT

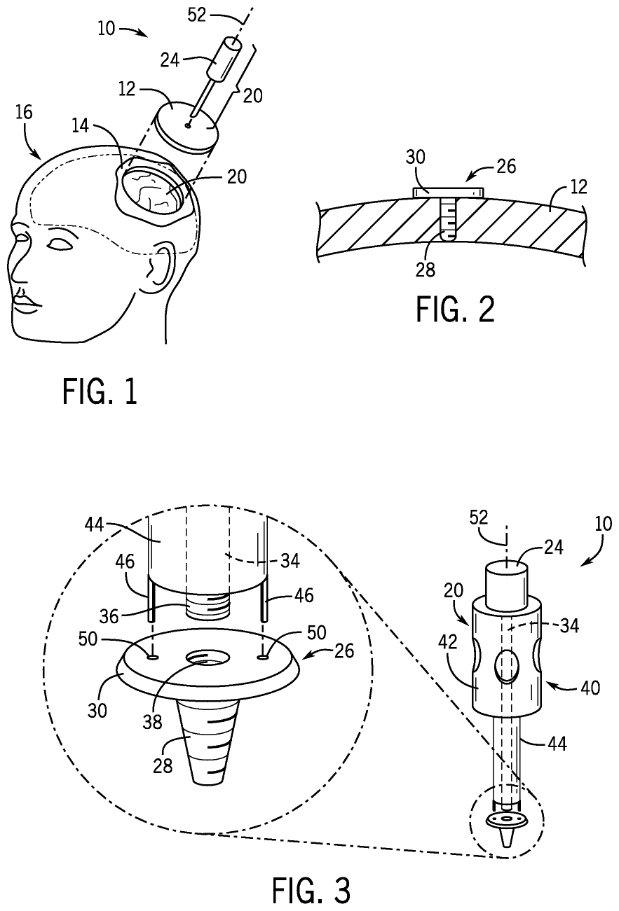

Referring now to FIG. 1, a surgical tool 10 per one embodiment of the present invention may be attached to an outer surface of a central location of a bone flap 12 such as may be removed from the skull 14 of the patient 16 during a craniotomy or the like.

The surgical tool 10 includes a handle portion 20 extending outwardly along an axis 52 generally normal to an outer surface of the bone flap 12 and fixed rotationally and angularly with respect to the bone flap 12 to provide a secure grip of the bone flap 12 through the handle portion 20. The handle portion 20 thus reduces the chance of dropping the bone flap 12 or having the sharp edges of the bone flap 12 tip into the patient's dura 22 or underlying brain tissue.

For this purpose the handle portion 20 may extend by a distance of at least 4 inches and typically 5 to 6 inches from the bone flap 12 and may terminate at an enlarged grip 24 having an increased diameter for easy grasping by a physician. The grip 24 may include a roughened or reduced-slip elastomeric surface. Generally the handle portion 20 will be sterilizable for a reuse.

Referring now to FIG. 2, the handle portion 20 may attach to the bone flap 12 by means of a screw 26 having a threaded portion 28 that may pass into a precut bore in the bone flap 12 for threaded engagement with the bone flap 12. For this purpose, the threads of the threaded portion 28 may be self-tapping.

A depth of insertion of the threaded portion 28 into the bone flap 12 is limited by a larger diameter-to-head portion 30 attached to the threaded portion 28 at a distal end of the threaded portion 28. The underside of the head portion 30 limits the depth of the insertion of the threaded portion 28 by abutting the outer surface of the bone flap 12 to limit protrusion of the threaded portion 28 beneath the bone flap 12 such as might injure underlying tissue. The proximal tip of the threaded portion 28 may be rounded for this purpose.

The length of the threaded portion will typically be less than 10 mm to conform with the thickness of the bone flap 12. The diameter of the threaded portion may be from 3 to 6 mm and the diameter of the head portion will typically be from 5 to 10 mm or less than 20 mm with an axial thickness of 2 to 3 mm.

In some embodiments, the screw 26 may be constructed of an x-ray transparent (radiolucent) material such as a polymer material, for example, polyester ether ketone (PEEK), to limit interference with computed tomography. Alternatively, the screw 26 may be a ceramic material compatible with magnetic resonance imaging. The invention contemplates that the material of the screw 26 will be sterilizable and exhibit long-term biocompatibility allowing the screw 26 to be retained in the bone flap 12 after surgery. In some embodiments, the screw 26 may be constructed of a metal such as titanium or stainless steel.

Referring now to FIG. 3, in one embodiment the handle portion 20 may provide a central, axial shaft 34 terminating at its proximal end in a threaded portion 36 that may be received by a corresponding threaded bore 38 through the head 30 of the screw 26 to retain the handle portion 20 against axial disconnection with the screw 26.

A torque driver 40 may coaxially surround the shaft 34 and provide a second grip 42 at its distal end and a tubular member 44 extending toward the bone flaps 12 and terminating at diametrically opposed axially extending spanner pins 46. The spanner pins 46 may be received within corresponding blind bores 50 in the upper surface of the screw head 30 to allow for the application of torque about the axis 52 of the tool 10 for inserting the screw 26 into the bone flap 12 and removing the same. This torque coupling provides a benefit over the threaded coupling of the threaded portion 36 and threaded bore 38 in resisting binding or unscrewing during some torquing applications. When the threaded portion 36 of the shaft 34 is fully engaged with the threaded bore 38, a corresponding axial engagement between the grip 24 and second grip 42 may retain the spanner pins 46 securely in the blind bores 50.

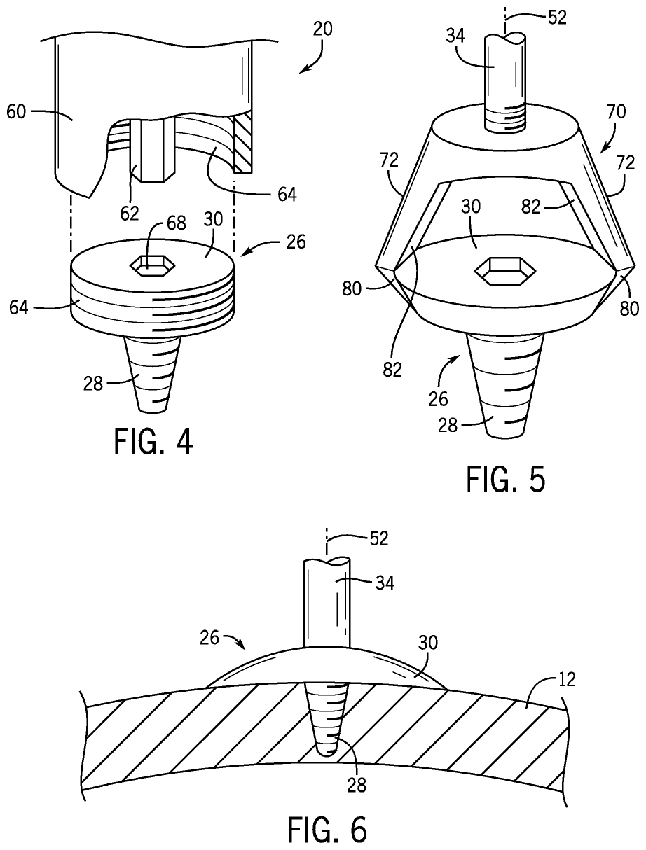

Referring now to FIG. 4, it will be appreciated that these functions of retention of the screw 26 on the handle portion 20 and torsion of the screw 26 by the torque driver 40 may be accomplished alternatively by providing an outer tubular shaft 60 communicating with the grip 24 (displaced downward to the location of the grip 42 as shown in FIG. 3) and providing a central hex drive shaft 62 communicating with the grip 42 (displaced upward to the position of the grip 24 with respect to the depiction of FIG. 3). Internal threads 64 on the proximal end of the tubular shaft 60 may then engage external threads 66 on the outer periphery of the head 30 of the screw 26 with the hex shaft 62 being received by a central hex bore 68. The engagement of the tubular shaft 60 with the external thread 66 retains the handle portion 20 on the screw 26 while the hex shaft 62 and the hex bore 68 provide for torque transmission.

Referring now to FIG. 5, the function of torque transmission and screw retention may be combined by means of a clamp head 70 having downwardly extending hook wings 72 terminating at inwardly extending hooks 80. The clamp head 70 may be slidingly engaged with the head 30 of the screw 26 in a lateral direction to axis 52, with the hooks 80 fitting underneath lips 74 formed at opposite sides of the head 30 of the screw 26. Flats 82 in the head 30 engaging the inner surface of the hook wings 72 allow for torque transmission while hooks 80 passing underneath the lips 74 of the screw 26 prevent axial disengagement.

Referring now to FIG. 5, in one embodiment, the invention may integrally attach the shaft 34 to the screw 26 to provide both retention and torque transmission eliminating the need for coaxial structures or separately assembled parts during a surgical procedure.

It will generally be appreciated that the handle portion 20 may be used manually or attached to a battery-powered drill or handle or the like.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. A surgical tool for stabilizing a bone flap during removal or attachment to a skull, the tool comprising:
a screw having a threaded portion terminating at a head portion, the threaded portion sized to be received through a bore in a central location in the bone flap to engage the bone flap to a depth controlled by a head portion abutting an outer surface of the bone flap; and
a stabilizing handle attaching to the screw to apply a torque to the screw to thread the threaded portion through the bore and to be retained on the screw to extend outwardly therefrom to a distal grip portion allowing stabilization of the bone flap during surgery.

2. The surgical tool of claim 1 wherein the depth controlled by the head portion is less than 10 mm.

3. The surgical tool of claim 2 wherein the threaded portion has an outer diameter from 3 to 6 mm.

4. The surgical tool of claim 1 wherein the screw is a radiolucent material.

5. The surgical tool of claim 4 wherein the screw is selected from the group consisting of a polymer and a ceramic.

6. The surgical tool of claim 1 wherein the head portion of the screw includes a second threaded portion attachable to corresponding threads of a proximal end of the stabilizing handle with rotation of the distal grip portion.

7. The surgical tool of claim 6 further including a drive engaging with the screw head and rotatable independently of the stabilizing handle for applying a torque to the screw.

8. The surgical tool of claim 7 wherein the drive and stabilizing handle are coaxial.

9. The surgical tool of claim 8 wherein the drive and screw engage at a connection selected from the group consisting of a hex pin and socket and a spanner pin and associated holes.

10. A method of stabilizing a bone flap during removal or attachment to a skull employing a stabilizing tool comprising:
a screw having a threaded portion terminating at a head portion, the threaded portion sized to be received through a bore in a central location in the bone flap to engage the bone flap to a depth controlled by a head portion abutting an outer surface of the bone flap; and
a stabilizing handle attaching to the screw to apply a torque to the screw to thread the threaded portion through the bore and to be retained on the screw to extend outwardly therefrom to a distal grip portion allowing stabilization of the bone flap during surgery; the method including:
(a) threading the screw through a bore in a central location of the bone flap;
(b) releasing the bone flaps from the skull while holding the stabilizing handle;
(c) replacing the bone flap in the skull while holding the stabilizing handle and affixing the bone flap to the skull; and
(d) removing the stabilizing handle.
